(19) United States
(12) Patent Application Publication
(10) Pub. No.: US 2008/0033191 A1
(43) Pub. Date: Feb. 7, 2008

(54) Process for producing glycerol having low aldehyde and ketone content and improved storage stability

(76) Inventors: Ulrich Schoerken, Duesseldorf (DE); David Melchior, Bergisch-Gladbach (DE); Jochen Seifert, Muehital (DE); Carolin Meyer, Duesseldorf (DE); Valerie Eychenne, Toulouse (FR); Christiane Richard-Elsner, Duesseldorf (DE); Horst Josten, Duesseldorf (DE); Jean Rigal, Tournefeuille (FR); Bernhard Gutsche, Hilden (DE); Albert Strube, Neuss (DE)

Correspondence Address: COGNIS CORPORATION;PATENT DEPARTMENT, 300 BROOKSIDE AVENUE, AMBLER, PA, 19002, US

(21) Appl. No.: 11/880,928

(22) Filed: Jul. 25, 2007

(30) Foreign Application Priority Data
Jul. 27, 2006 (DE) 10 2006 034 702.1

(51) Int. Cl. C07C 51/00

(52) U.S. Cl. 554/124

(57) ABSTRACT

The invention describes a process for the production of glycerol of providing a vegetable oil or fat as starting material, obtaining crude glycerol from the vegetable oil or fat, and treating the crude glycerol with a reducing agent. The present invention also describes a process for the production of a medicament or pharmaceutical composition, the medicament or composition formed by combining glycerol and at least one pharmaceutically active ingredient. The present invention also describes a pharmaceutical composition or medicament composed of glycerol obtained by way of the invention and a pharmaceutically active ingredient, which compositions forms very little glyceraldehyde and dihydroxyacetone during storage.

### CROSS REFERENCE TO RELATED APPLICATIONS

The present application claims priority under 35 U.S.C. Section 119 of German Application No. 102006034702.1 filed Jul. 27, 2006, the contents of which are incorporated herein in its entirety.

### FIELD OF THE INVENTION

This invention relates to a process for the production of glycerol comprising providing a vegetable oil or fat as starting material, obtaining crude glycerol from the vegetable oil or fat and treating the crude glycerol with a reducing agent. The present invention also relates to a process for the production of a medicament or pharmaceutical composition, the medicament containing glycerol and at least one pharmaceutically active ingredient and the process for producing a medicament comprising the above-mentioned process for producing glycerol and, additionally, the step of contacting the glycerol with at least one pharmaceutically active ingredient. The present invention also relates to glycerol and a medicament obtainable by the above-mentioned processes and to glycerol and a medicament which form very little glyceraldehyde and dihydroxyacetone during storage.

### BACKGROUND OF THE INVENTION

Glycerol is important, inter alia, as a raw material for medicaments. Glycerol can be produced from vegetable oils or fats, from animal oils or fats and synthetically from propylene.

Synthetically produced glycerol obtained from propylene is generally used for pharmaceutical applications which require high-purity glycerol. Glycerol based on vegetable or animal fats generally does not have the requisite quality.

Vegetable glycerol can be obtained from various sources, for example from pulp oils, such as palm or olive oil, or from seed oils, such as soybean oil, rapeseed oil, sunflower oil, thistle oil, linseed oil, peanut oil, coconut oil or palm kernel oil.

There are various processes which convert vegetable or animal fats or oils into glycerol and fatty acids or esters of fatty acids. Standard methods include, for example, the high-pressure splitting of oils, high-pressure transesterification with methanol in the presence of zinc catalysts and alkaline low-pressure transesterification with methanol. More recent alternative methods, which have not yet been used on an industrial scale, include, for example, enzymatic hydrolysis and the transesterification of vegetable oils and low-pressure transesterification with phase-transfer catalysts. In connection with these processes, people skilled in the art normally refer to an oil phase and a glycerol phase. The oil phase is either the fatty acid phase obtainable by oil splitting or the methyl ester phase obtainable by transesterification. The glycerol phase settles out below the oil phase and can be removed, for example, by decantation.

For further purification, glycerol can be separated from the oil phase and then further purified by various methods. Separators are mostly used for removing the oil phase, in which case the removal of oil residues (oil residues means either fatty acid or methyl esters and some (partial) glycerides from incomplete reaction of the oil) can be carried out by pH shift and settling tanks. Methods for further purification used on an industrial scale include, for example, fractional distillation, chromatographic methods, such as ion exclusion chromatography for removing salts and the use of resins, such as ion exchangers or amine resins, for the selective binding of secondary products. In addition, absorptive methods, such as treatments with active carbon or bleaching earth, can be carried out to remove coloring and odorous substances.

The glycerol produced varies in quality according to the vegetable oil or vegetable fat or the animal oil or fat used, according to the industrial oil processing method used, and according to the combination used to purify the glycerol (cf. also the Examples of the present specification). In storage tests, the glycerol purified by standard methods lacks oxidation stability and the formation of aldehydes and ketones can be clearly detected.

Glycerol can contain aldehydes and ketones as an impurity. If they come into contact with proteins, aldehydes and ketones can form Schiff's bases with the amine groups of proteins and can thus modify and, in some cases, even crosslink the proteins. This is a particular disadvantage where glycerol is used as a raw material for medicaments, especially those which contain protein, or where the medicaments come into contact with proteins in the human body. Accordingly, it is important, especially for pharmaceutical applications, to produce glycerol with a minimal content of aldehydes and ketones. In addition, the glycerol—if possible—should not form any aldehydes or ketones (for example by oxidation) during storage.

EP-B 1 242 121 (Eli Lilly) discloses glycerol with a low content of aldehydes and pharmaceutical compositions containing this glycerol. The glycerol can be of vegetable origin or can have been synthetically produced from propylene. The document also discloses a process for producing this glycerol in which the aldehyde content of the glycerol is reduced by polymer resins containing amino groups.

The problem addressed by the present invention was to provide a process for the production of glycerol, which process would produce a glycerol having a low content of aldehydes and ketones and which glycerol would also be stable in storage. “Stable in storage” means that there would be only minimal formation of aldehydes or ketones during storage of the glycerol.

### BRIEF SUMMARY OF THE INVENTION

The problem stated above has been solved by the process of the present invention for the production of glycerol comprising providing a vegetable oil or fat as starting material, obtaining crude glycerol from the vegetable oil or fat and treating the crude glycerol with a reducing agent, preferably with a hydrogenating agent, preferably with a borohydride, more particularly with sodium borohydride.

Accordingly, one aspect of the invention is a process for producing glycerol having a low content of aldehydes and ketones and having improved storage stability comprising:
(a) providing a crude glycerol obtained from a vegetable oil or fat;
(b) treating the crude glycerol with a reducing agent; and
(c) producing glycerol having an aldehyde and ketone content of less than 9 ppm and having improved storage stability.

Another aspect of the invention is a glycerol obtained by the process of the invention, which glycerol has a lower content of aldehydes and ketones and improved storage stability properties over glycerol formed by other processes.

The glycerol produced by way of the invention has improved storage properties over glycerol produced by other processes. After storage in the dark at 40° C. for a period of 8 weeks, the glycerol has a total content of glyceraldehyde and dihydroxyacetone of less than 9 ppm.

Yet another aspect of the invention is the glycerol produced by way of the present invention in combination with a pharmaceutically active ingredient to form a medicament or pharmaceutical composition having the improved properties of the glycerol achieved by way of the invention as a component of the medicament or composition.

Additional aspects of the invention include the following embodiments:
The process for the production of glycerol further comprising distillation (preferably fractional distillation) of the crude glycerol before it is treated with the reducing agent.
The process for the production of glycerol, the reducing agent being a borohydride, further comprising removal of the boron (in the form of a boron compound) from the glycerol by distillation after treatment with the borohydride.
The process for the production of glycerol, the crude glycerol being obtained from the vegetable oil or fat at temperatures below 100° C. and under pressures below 2 bar (so-called low temperature/low pressure process).
Glycerol obtainable by the process according to the invention, more particularly glycerol obtainable by the process according to the invention in which the content of glyceraldehyde and dihydroxyacetone (in total) after storage of the glycerol in darkness in standard transparent glass bottles for laboratory chemicals over a period of 8 weeks at 40° C. is less than 9 ppm, preferably less than 6 ppm, more preferably less than 3 ppm and most preferably less than 2 ppm, the content of glyceraldehyde and dihydroxyacetone (in total) being determined by HPLC (high-performance liquid chromatography), the HPLC determination being carried out in particular under the conditions set out in Example 1B of the present specification.
Glycerol in which the content of glyceraldehyde and dihydroxyacetone (in total) after storage of the glycerol in darkness in standard transparent glass bottles for laboratory chemicals over a period of 8 weeks at 40° C. is less than 9 ppm, preferably less than 6 ppm, more preferably less than 3 ppm, and most preferably less than 2 ppm, the content of glyceraldehyde and dihydroxyacetone (in total) being determined by HPLC (high-performance liquid chromatography), the HPLC determination being carried out in particular under the conditions set out in Example 1B of the present specification.
A process for the production of a medicament, the medicament containing glycerol and at least one pharmaceutically active ingredient; the process comprising the producing of glycerol by way of the invention and contacting or combining the glycerol with at least one pharmaceutically active ingredient.
The process for the production of a medicament, the medicament containing a protein or the medicament containing a compound which contains amino groups.
A medicament obtainable by the process according to the invention for the production of a medicament, the glycerol being in particular a glycerol in which the content of glyceraldehyde and dihydroxyacetone (in total) after storage of the glycerol in darkness in standard transparent glass bottles for laboratory chemicals over a period of 8 weeks at 40° C. is less than 9 ppm, preferably less than 6 ppm, more preferably less than 3 ppm, and most preferably less than 2 ppm, the content of glyceraldehyde and dihydroxyacetone (in total) being determined by HPLC (high-performance liquid chromatography), the HPLC determination being carried out in particular under the conditions set out in Example 1B of the present specification.
A medicament containing the glycerol according to the invention and at least one pharmaceutically active ingredient and, more particularly, additionally containing a protein or a compound containing amino groups.

### BRIEF DESCRIPTION OF THE DRAWINGS

FIG. 1 illustrates an apparatus for purifying glycerol in which the crude glycerol is treated with borohydride in a batch process.

FIG. 2 illustrates an apparatus for purifying glycerol in which the crude glycerol is treated with borohydride and silicate combined in a batch process.

FIG. 3 illustrates an apparatus for purifying glycerol in which the crude glycerol is treated with borohydride in the liquid phase.

FIG. 4 illustrates an apparatus for purifying glycerol in which the crude glycerol is treated with borohydride in the gas phase coupled with distillation.

### DETAILED DESCRIPTION OF THE INVENTION

The glycerol according to the invention and the medicament according to the invention as defined in the preceding paragraphs differ from the prior art in their higher stability in storage, i.e. in the minimal formation of glyceraldehyde and dihydroxyacetone during storage.

The process according to the invention for the production of glycerol has many advantages. It can be carried out in such a way that the glycerol produced by the process according to the invention satisfies the requirements for pharmaceutical raw materials as laid down in the standard work relevant to the pharmaceutical industry, namely the Pharmacopoeia.

If, in the process according to the invention for the production of glycerol, the crude glycerol is purified by distillation before the treatment with a reducing agent, a particularly pure and storage-stable glycerol can be obtained with minimal need for reducing agent.

Glycerol of vegetable origin obtainable by the process according to the invention is more stable in storage than glycerol from propylene subjected to a reducing step. This is illustrated in particular by the Examples of the present specification.

Glycerol with a very low content of aldehydes and ketones can be produced by the process according to the invention. Storage-stable glycerol can also be produced. Storage stability shall be understood to mean, in particular, high oxidation stability during storage, i.e. there is no significant formation of aldehydes or ketones during storage of the glycerol. Storage over a period of 8 weeks may be used as the standard in this regard.

It can be of advantage to use suitable raw materials in the process according to the invention, more particularly vegetable fats or oils which contribute towards a low content of aldehydes and ketones in the glycerol obtained.

It can be of advantage in the process according to the invention to obtain the crude glycerol from the vegetable fat or oil, which can be carried out by known methods, by a process which contributes towards a low content of aldehydes and ketones in the glycerol obtained.

It can be of advantage in the process for obtaining crude glycerol from the vegetable fat or oil to provide purification steps (for example distillation, more particularly fractional distillation) which contribute towards a low content of aldehydes and ketones in the glycerol obtained.

It can be of advantage to provide other purification steps besides reduction in the process for obtaining crude glycerol from the vegetable fat or oil. Such purification steps include, in particular, purification with amine-containing compounds which react chemically with contaminating aldehydes and ketones. The amine-containing compounds may be aminofunctionalized resins, although a reaction with formaldehyde or diamines or a reaction with alkylamines, such as stearyl amine, followed by removal of the oil-soluble amine by extraction may also be carried out. Adsorption, for example onto adsorber resins or active carbon, is another possibility, as is catalytic oxidation of the aldehydes to carboxylic acids and the ketones to carboxylic acids with cleavage of the carbon chain. These processes contribute towards a low content of aldehydes and ketones in the glycerol obtained.

The glycerol produced by the process according to the invention is particularly suitable for pharmaceutical applications and for use in pharmaceutical compositions, more particularly for use in mixtures with proteins or other amine-containing compounds, in particular for parenteral administration.

The process according to the invention utilizes a vegetable oil or fat. This vegetable oil or fat may be, in particular, sunflower oil or soybean oil or low-erucic rapeseed oil. Low-erucic rapeseed oil is rapeseed oil from a rape variety which has a low erucic acid content (<1% by weight) and which is used, for example, in nutrition. The corresponding high-erucic rapeseed oil is normally used in technical applications.

Obtaining crude glycerol from the vegetable oil or fat can be carried out by known methods. These may be, in particular, low-pressure processes and low-temperature processes. More particularly, alkaline low-pressure transesterification, transesterification through phase-transfer catalysis, transesterification through enzymatic catalysis or enzymatic hydrolysis may be used. So-called low-pressure/low-temperature processes, i.e. processes carried out at temperatures below 100° C. and at pressure below 2 bar (absolute pressure), are preferred. The following processes are suitable for obtaining the crude glycerol:
alkaline low-pressure transesterification: temperature ca. 80° C., pressure <2 bar, catalyst sodium or potassium methylate, 2- or 3-stage process with removal of glycerol in the process;
phase-transfer catalysis: typical conditions: 60° C., normal pressure, catalyst, for example quaternary amine compounds (for example ALIQUAT® quaternary amine compounds from Cognis Deutschland GmbH & Co. KG, Düsseldorf, Germany);
enzymatic catalysis: typical conditions: 30-60° C., normal pressure, catalyst lipase in free or immobilized form.

Crude glycerol in the context of the present invention is any glycerol before its treatment with a reducing agent. Accordingly, crude glycerol can be glycerol of various purities. For example, crude glycerol can be glycerol which is obtained directly, i.e. without further purification steps, from the splitting of fats. Crude glycerol can be glycerol which has already been subjected to purification steps, for example to distillation. Particularly, crude glycerol in the context of the present invention can be glycerol which satisfies the purity requirements for pharmaceutical applications to EP/USP (so-called EP/USP pharma-grade glycerol).

The process according to the invention for the production of glycerol can comprise further working-up or purification steps. These steps may be carried out either before or after the treatment of the crude glycerol with a reducing agent. More particularly, the crude glycerol may be subjected to fractional distillation, more especially before its treatment with a reducing agent. In addition, the crude glycerol may be subjected to a combination of distillation, more especially fractional distillation, and a treatment with active carbon. The crude glycerol may be subjected to this combined treatment in particular before it is treated with a reducing agent.

The process according to the invention for the production of glycerol encompasses treating the crude glycerol with a reducing agent. Suitable reducing agents are metal hydrides such as, for example, LiAlH₄, NaBH₄ and others, molecular hydrogen with metal catalysis using, for example, nickel and platinum; reducing inorganic nitrogen, sulfur and phosphorus compounds, such as hypophosphorous acid for example. The reducing agent may be, in particular, a hydrogenating agent, more particularly a borohydride, more especially sodium borohydride. The borohydride may be used, in particular, in solution in an alkaline liquid formulation. In one particular embodiment, the reducing step (more particularly with borohydride) may be followed by distillation (more particularly fractional distillation, also molecular distillation with the briefest exposure of the glycerol to heat). In addition, the reducing step may be preceded or followed by treatment of the glycerol or crude glycerol with silicates. The treatment of the glycerol or crude glycerol with silicates can remove traces of metals.

Treatment with a borohydride may be carried out as follows:

The quantity of borohydride solution used may be, for example, 100 to 5,000 ppm and more particularly 250 to 2,000 ppm, based on the crude glycerol to be treated. The temperature during the treatment may be in the range from 50 to 90° C. The pH value during the treatment may be 7 or higher. The treatment time may be 2 minutes to 12 hours. The treatment may be carried out in a nitrogen atmosphere. Any excess borohydride remaining can be removed by acidification and/or by distillation, more particularly by distillation. With acidification, borate is obtained in the product which is often undesirable. Because of this, the boron is often removed by distillation or, alternatively, by adsorption onto a borate-specific adsorber.

The following variations may be made for carrying out the process according to the invention:
a batch process after fractional distillation with timed reactors
a continuous process after fractional distillation in a tube reactor with mixers
a continuous process with a borohydride fixed bed in the gas phase installed between a first thin-layer evaporator and a fractionating column.

In addition, the process according to the invention may comprise a silicate treatment which may be carried out as follows:
the quantity of silicate used may be from 100 to 10,000 ppm and, more particularly, from 500 to 5,000 ppm, based on the glycerol or crude glycerol to be treated
the treatment temperature may be in the range from 50 to 90° C.
the treatment may be carried out in combination with a borohydride treatment either simultaneously or in succession.

When the process according to the invention includes a distillation step, the distillation step may be carried out as follows:
as thin-layer distillation or in a falling-film evaporator, more particularly with brief heat stress
under a pressure below 2 mbar
at a temperature below 180° C.
distillation may be followed by expansion with nitrogen
further storage and packaging may be carried out under nitrogen.

The content of aldehydes in glycerol is normally carried out to the Pharmacopoeia standard using pararosaniline hydrochloride. Now, it has been shown by way of the present invention that this test is highly specific for formaldehyde, but does not quantitatively analyze for the presence of other aldehydes, which are oxidation products of glycerol (for example glyceraldehyde or dihydroxyacetone), cf. Examples of the present specification.

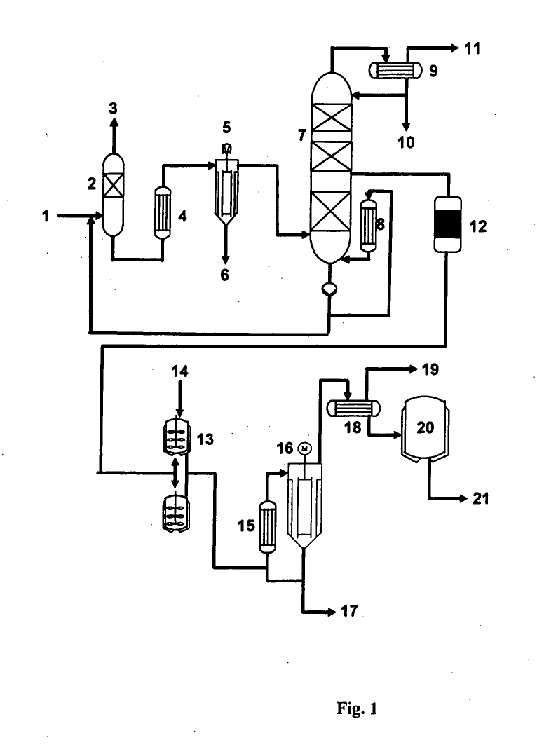

FIG. 1 illustrates an apparatus for purifying glycerol. In this apparatus, crude glycerol can be treated with borohydride in a batch process. The reference numerals illustrate the apparatus as follows:
1: crude glycerol after removal of the oil phase
2: dryer
3: vacuum unit, separation of low boilers/water
4: heating system
5: thin-layer evaporator
6: bottom product (salts+high boilers)
7: fractionating column
8: reboiler system
9: condensation system
10: head product (low boilers)
11: vacuum system
12: active carbon treatment (either as a column or as a batch reactor)
13: 2 stirred-tank reactors for the borohydride treatment (timed)
14: addition of borohydride
15: heating system
16: thin-layer evaporator (or falling-film evaporator)
17: bottom product (high boilers)
18: condensation system
19: vacuum system
20: storage tank for pure glycerol
21: bottling.

Using the described apparatus, an aldehyde- and ketone-free glycerol can be produced which has a shelf life of >8 weeks with no reformation of aldehydes and ketones.

Alternatively, after the borohydride treatment 13, the product can be taken back through the thin-layer evaporator 5 and the head product condensed. In this case, the stream flowing into the thin-layer evaporator 5 can be switched at intervals from crude glycerol to purified glycerol. This variant requires another buffer tank. Components 15 to 19 of the apparatus are then not utilized in this process.

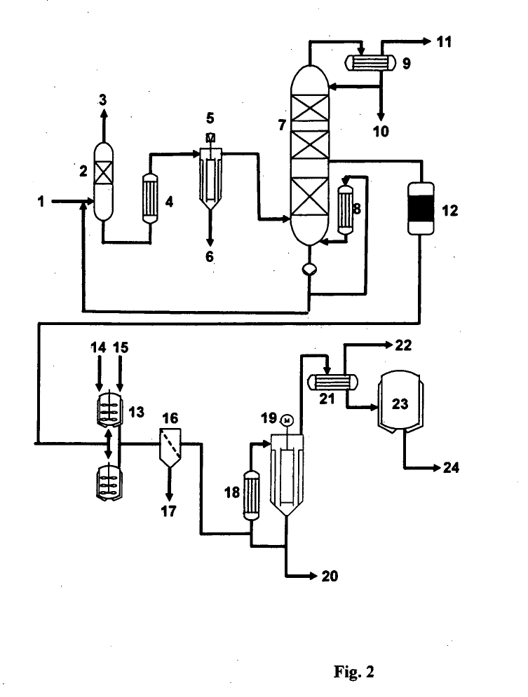

FIG. 2 illustrates an apparatus for purifying glycerol. In this apparatus, crude glycerol can be treated with borohydride and silicate combined in a batch process. The reference numerals illustrate the apparatus as follows:
1: crude glycerol after removal of the oil phase
2: dryer
3: vacuum unit, separation of low boilers/water
4: heating system
5: thin-layer evaporator
6: bottom product (salts+high boilers)
7: fractionating column
8: reboiler system
9: condensation system
10: head product (low boilers)
11: vacuum system
12: active carbon treatment (either as a column or as a batch reactor)
13: 2 stirred-tank reactors for the borohydride and silicate treatment (timed)
14: addition of borohydride
15: addition of silicate
16: silicate filtration
17: solid waste
18: heating system
19: thin-layer evaporator (or falling-film evaporator)
20: bottom product (high boilers)
21: condensation system
22: vacuum system
23: storage tank for pure glycerol
24: bottling.

Using the described apparatus, an aldehyde- and ketone-free glycerol can be produced which has a shelf life of >8 weeks with no re-formation of aldehydes and ketones.

Alternatively, after the silicate filtration 16, the product can be taken back through the thin-layer evaporator 5 and the head product condensed. In this case, the stream flowing into the thin-layer evaporator 5 can be switched at intervals from crude glycerol to purified glycerol. This variant requires another buffer tank. Components 18 to 22 of the apparatus are then not utilized in this process.

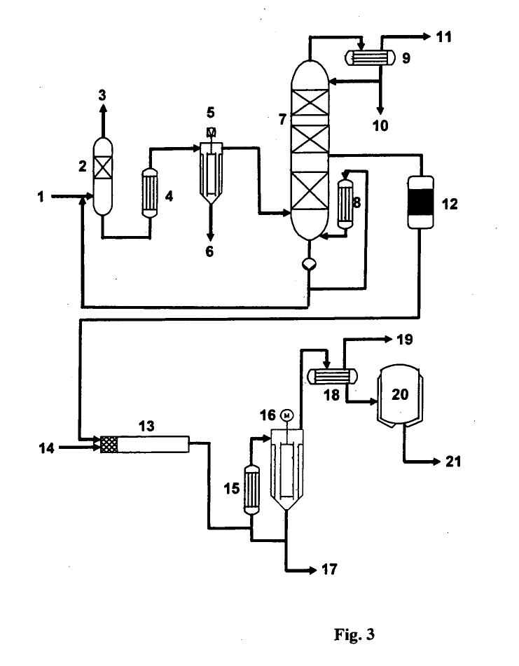

FIG. 3 illustrates an apparatus for purifying glycerol. In this apparatus, crude glycerol can be continuously treated with borohydride in the liquid phase. The reference numerals illustrate the apparatus as follows:
1: crude glycerol after removal of the oil phase
2: dryer
3: vacuum unit, separation of low boilers/water
4: heating system
5: thin-layer evaporator
6: bottom product (salts+high boilers)
7: fractionating column
8: reboiler system
9: condensation system
10: head product (low boilers)
11: vacuum system
12: active carbon treatment (either as a column or as a batch reactor)
13: tube reactor for borohydride reduction with built-in micromixer or alternatively with built-in static mixers
14: addition of borohydride
15: heating system
16: thin-layer evaporator (or falling-film evaporator)
17: bottom product (high boilers)
18: condensation system
19: vacuum system
20: storage tank for pure glycerol
21: bottling.

Using the described apparatus, an aldehyde- and ketone-free glycerol can be produced which has a shelf life of >8 weeks with no re-formation of aldehydes and ketones.

Alternatively, after the borohydride treatment 13, the product can be taken back through the thin-layer evaporator 5 and the head product condensed. In this case, the stream flowing into the thin-layer evaporator 5 is switched at intervals from crude glycerol to purified glycerol. This variant requires another buffer tank. Components 15 to 19 of the apparatus are then not utilized in the process.

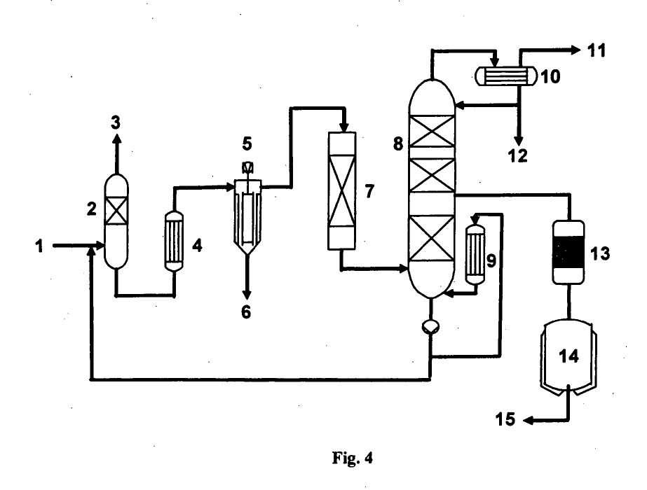

FIG. 4 illustrates an apparatus for purifying glycerol. In this apparatus, crude glycerol can be treated with borohydride in the gas phase coupled with distillation. The reference numerals illustrate the apparatus as follows:
1: crude glycerol after removal of the oil phase
2: dryer
3: vacuum unit, separation of low boilers/water
4: heating system
5: thin-layer evaporator
6: bottom product (salts+high boilers)
7: reactor for fixed-bed borohydride treatment: glycerol is passed through the fixed bed in the gas phase
8: fractionating column
9: reboiler system
10: condensation system
11: vacuum system
12: head product (low boilers)
13: active carbon treatment (either as a column or as a batch reactor)
14: storage tank for pure glycerol
15: bottling.

Using the described apparatus, an aldehyde- and ketone-free glycerol can be produced which has a shelf life of >8 weeks with no re-formation of aldehydes and ketones.

In this apparatus, in contrast to the apparatus shown in FIGS. 1 to 3, the borohydride treatment is carried out in partly purified glycerol. In this process, the borohydride concentration used is higher.

The apparatus is preferably operated continuously with two borohydride fixed beds. One of the fixed beds is in operation mode, while the borohydride filling in the other fixed bed is replaced.

The following examples are illustrative of the invention and should not be construed in any manner whatsoever as limiting the scope of the invention.

### EXAMPLES

### Example 1

### Comparative Glycerol Analysis

A) Pararosaniline Stain Test

Formaldehyde, propionaldehyde, glyceraldehyde, hydroxyacetone and dihydroxyacetone were tested for their detectability at various concentrations by the pararosaniline hydrochloride stain test according to the Pharmacopoeia:

| | Formal- | Propion- | Glycer- | Hydroxy- | Dihydroxy- |
|---|---|---|---|---|---|
| Sub- | dehyde | aldehyde | aldehyde | acetone | acetone |
| stance | Abs. | Abs. | Abs. | Abs. | Abs. |
| ppm | 552 nm | 552 nm | 552 nm | 552 nm | 552 nm |
| 0 | 0 | 0 | 0 | 0 | 0 |
| 3 | 0.06 | nd | nd | nd | nd |
| 6 | 0.14 | nd | nd | nd | nd |
| 9 | 0.29 | nd | nd | nd | nd |
| 15 | 0.74 | nd | nd | nd | nd |
| 30 | 3.8 | 0.07 | 0.02 | 0.02 | 0.02 |
| 100 | >Limit | 0.99 | 0.04 | 0.02 | 0.02 |
| 300 | >Limit | >Limit | 0.18 | 0.02 | 0.02 |

In the above Table:
Abs. = absorption (at a wavelength of 552 nm in the photometer)
Limit = the absorption limit of the photometer which is 4
nd = not determined

Result:

The stain test is highly sensitive only for formaldehyde. The test is not suitable for detecting other aldehydes and ketones often present in glycerol, such as for example the direct oxidation products glyceraldehyde and dihydroxyacetone and the hydroxyacetone obtainable by rearrangement.

B) HPLC Analysis

Various aldehydes and ketones were derivatized with 2,4-dinitrophenyl hydrazine and the hydrazone compounds formed were separated by HPLC in a phosphoric acid/acetonitrile gradient and analyzed by a diode array at 340 nm. A reversed phase column (Reprosil Pur C18 AQ) was used for the separation. The following detection limits were determined.

| Substance | Detection limit |
|---|---|
| Formaldehyde | 0.5 |
| Acetaldehyde | 0.5 |
| Acrolein | 0.5 |
| Acetone | 0.5 |
| Propanal | 0.5 |
| Butanal | 0.5 |
| Glyceraldehyde | 2 |
| Dihydroxyacetone | 2 |
| Hydroxyacetone | 2 |
| Malondialdehyde | 10 |
| Glyoxal/benzaldehyde | 10 |
| Hexanal | 10 |
| Octanal | 10 |
| Decanal | 10 |
| Dodecanal | 10 |
| Tetradecanal | 10 |
| Hexadecanal | 10 |

Result:

The HPLC method is very suitable for analyzing various aldehydes and ketones in glycerol in traces. HPLC analysis was used for all other tests.

### Example 2

### Comparison of Crude Glycerol Quality Produced by Various Oleochemical Processes

Various crude glycerols were analyzed for their contents of aldehydes and ketones. Glycerols from enzymatic hydrolysis (sample A, pilot scale); from high-pressure oil splitting (sample B, pilot scale); from alkaline low-pressure transesterification (sample C, production scale); from zinc-catalyzed high-pressure transesterification (sample D, production scale) and from catalyst-free high-pressure and high-temperature transesterification (sample E, pilot scale) were analyzed. The symbol “<” means below the corresponding detection limit as specified in Example 1.

| | A | B | C | D | E |
|---|---|---|---|---|---|
| Substance | ppm | ppm | ppm | ppm | ppm |
| Formaldehyde | 0.6 | 4.9 | 6.7 | 12.2 | 130 |
| Acetaldehyde | < | 1.1 | 0.7 | 2.4 | 13 |
| Acrolein | 2.5 | 1.8 | < | 1.3 | 9 |
| Acetone | < | 0.6 | < | < | 14 |
| Propanal | 2.9 | 6.2 | 2.2 | 0.7 | 9.6 |
| Butanal | < | 5.8 | < | 0.9 | 1.2 |
| Glyceraldehyde (GA) | 2.8 | 12 | 9 | 5 | 60 |
| Dihydroxyacetone (DHA) | 2.5 | 18 | 6 | 8 | 120 |
| Hydroxyacetone (HA) | 2.3 | 51 | < | 18 | 420 |
| Malondialdehyde | < | 40 | < | < | 120 |
| Glyoxal/benzaldehyde | < | 28 | < | 11 | 260 |
| Hexanal | < | < | < | < | < |
| Octanal | < | < | < | < | < |
| Decanal | < | < | < | < | < |
| Dodecanal | < | < | < | < | < |
| Tetradecanal | < | < | < | < | < |
| Hexadecanal | < | < | < | < | < |
| Total aldehydes/ketones | 13.6 | 169.4 | 24.6 | 59.5 | 1156.8 |
| Total GA, DHA, HA | 7.6 | 81 | 15 | 31 | 600 |

Result:

Glycerols from the processes carried out at low temperatures and in the absence of pressure have the best starting quality.

### Example 3

### Comparison of the Crude Glycerol Quality of Various Vegetable Raw Material Sources

Crude glycerols based on various vegetable oils were reacted by alkaline low-pressure transesterification on a production scale and the glycerols were analyzed immediately after splitting (A samples) and after removal of methanol and defatting (B samples). The following raw material sources were analyzed: high erucic rapeseed oil (1A+1B), low erucic rapeseed oil (2A+2B) and palm oil (3A+3B). The symbol “<” means below the corresponding detection limit as specified in Example 1.

| | 1A | 1B | 2A | 2B | 3A | 3B |
|---|---|---|---|---|---|---|
| Substance | ppm | ppm | ppm | ppm | ppm | ppm |
| Formaldehyde | 2.8 | 0.5 | 4.5 | 0.7 | 4.9 | 0.9 |
| Acetaldehyde | 6.4 | 0.7 | 4.7 | 0.7 | 9.2 | 1.1 |
| Acrolein | < | < | < | < | < | < |
| Acetone | 6 | < | 4.1 | < | 12.8 | < |
| Propanal | 10.4 | 1.8 | 7.2 | 0.7 | 12.4 | 3 |
| Butanal | 2.4 | < | 1.4 | < | 3.2 | < |
| Glyceraldehyde (GA) | 3 | 4 | 4 | 6 | 24 | 6 |
| Dihydroxyacetone (DHA) | < | 3 | 2 | 4 | 10 | 7 |
| Hydroxyacetone (HA) | 2 | 11 | < | < | 4 | < |
| Malondialdehyde | 21 | < | < | < | 15 | < |
| Glyoxal/benzaldehyde | < | < | < | < | 16 | < |
| Hexanal | < | < | < | < | 10 | < |
| Octanal | < | < | < | < | < | < |
| Decanal | < | < | < | < | < | < |
| Dodecanal | < | < | < | < | < | < |
| Tetradecanal | < | < | < | < | < | < |
| Hexadecanal | < | < | < | < | < | < |
| Total aldehydes/ketones | 54 | 21 | 27.9 | 12.1 | 121.5 | 18 |
| Total GA, DHA, HA | 5 | 18 | 6 | 10 | 38 | 13 |

Result:

Low erucic rapeseed oil is very suitable as a raw material source. Palm oil as a raw material source leads to the highest concentration of contaminating aldehydes and ketones.

### Example 4

### Comparison of the Crude Glycerol Quality from Various Purification Processes

A comparison was made of the aldehyde and ketone contents of pure glycerols chemically produced from propylene (sample A), vegetable glycerol purified by fractional distillation (samples B+C, two separate production-scale batches) and vegetable glycerol purified using ion exchangers and absorptive resins (sample D, production scale). All the glycerols studied satisfied the Pharmacopoeia guidelines.

| | A | B | C | D |
|---|---|---|---|---|
| Substance | ppm | ppm | ppm | ppm |
| Formaldehyde | < | < | < | 5 |
| Acetaldehyde | < | < | < | < |
| Acrolein | < | < | < | < |
| Acetone | < | < | < | < |
| Propanal | < | < | < | < |
| Butanal | < | < | < | < |
| Glyceraldehyde (GA) | < | < | 2.5 | 8.3 |
| Dihydroxyacetone (DHA) | < | < | < | 10 |
| Hydroxyacetone (HA) | < | < | < | 1.9 |
| Malondialdehyde | < | < | < | 15 |
| Glyoxal/benzaldehyde | < | < | < | < |
| Hexanal | < | < | < | < |
| Octanal | < | < | < | < |
| Decanal | < | < | < | < |
| Dodecanal | < | < | < | < |
| Tetradecanal | < | < | < | < |
| Hexadecanal | < | < | < | < |
| Total aldehydes/ketones | 0 | 0 | 2.5 | 40.2 |
| Total GA, DHA, HA | 0 | 0 | 2.5 | 20.2 |

Result:

Glycerol purified by fractional distillation has a high quality comparable with that of glycerol produced chemically from propylene. By contrast, glycerol purified by chromatography still contains aldehyde and ketone impurities which are not quantitatively detected by the pararosaniline hydrochloride stain test.

### Example 5

### Hydrogenation and Distillation of Crude Glycerol

30 g water and 0.6 g of an alkaline borohydride solution with a borohydride content of 20% by weight were added to 600 g of the various crude glycerols, followed by incubation under nitrogen for 2 hours at 80° C. The water was then distilled off in vacuo and the dried glycerols were subjected to short-path distillation at a temperature of 150° C. The distillation apparatus was purged with nitrogen and the glycerols were bottled under nitrogen. Crude glycerols from enzymatic hydrolysis (Example 2, sample A) and from alkaline low-pressure transesterification (Example 2, sample C) were used for the tests. The samples were subjected to a storage test (see following Examples).

Result:

The aldehyde and ketone levels in the samples were distinctly reduced. Hydrogenation was more successful with the glycerol from enzymatic hydrolysis. The reason for this was a low concentration of impurities which consume the borohydride.

### Example 6

### Hydrogenation and Distillation of Pure Glycerol

30 g water and 0.6 g of an alkaline borohydride solution with a borohydride content of 20% by weight were added to 600 g of the various pure glycerols, followed by incubation under nitrogen for 2 hours at 80° C. The water was then distilled off in vacuo and the dried glycerols were subjected to short-path distillation at a temperature of 150° C. The distillation apparatus was purged with nitrogen and the glycerols were bottled under nitrogen. Pure glycerols from Example 4, Samples B and C, were used for the tests. The samples were subjected to a storage test (see following Examples).

Result:

No aldehydes or ketones could be detected by HPLC in either sample.

### Example 7

### Hydrogenation and Distillation of Pure Glycerol

30 g water and 0.6 g of an alkaline borohydride solution with a borohydride content of 20% by weight were added to 600 g pure glycerol (sample B from Example 4), followed by incubation under nitrogen for 2 hours at 80° C. After the reaction, excess borohydride was destroyed by acidification with hydrochloric acid to pH 5. The water was then distilled off in vacuo and the dried glycerols were subjected to short-path distillation at a temperature of 150° C. The distillation apparatus was purged with nitrogen and the glycerols were bottled under nitrogen. The sample was subjected to a storage test (see following Examples).

Result:

No aldehydes or ketones could be detected by HPLC in either sample.

### Example 8

### Comparison of the Glycerol Quality of the Hydrogenated and Distilled Glycerols

The samples prepared in Examples 5, 6 and 7 were compared with the corresponding starting substances: glycerol from Example 2, sample A; B: sample A hydrogenated (Example 5); C: glycerol from Example 2, sample C; D: sample C hydrogenated (Example 5); E: glycerol from Example 4, sample B; F: sample E hydrogenated (Example 6); G: sample E hydrogenated and neutralized (Example 7); H: glycerol from Example 4, sample C; I: sample H hydrogenated (Example 6).

| | A | B | C | D | E |
|---|---|---|---|---|---|
| Substance | ppm | ppm | ppm | ppm | ppm |
| Formaldehyde | 0.6 | < | 6.7 | 1.5 | < |
| Acetaldehyde | < | < | 0.7 | 1.1 | < |
| Acrolein | 2.5 | < | < | < | < |
| Acetone | < | < | < | 0.6 | < |
| Propanal | 2.9 | < | 2.2 | 0.6 | < |
| Butanal | < | < | < | < | < |
| Glyceraldehyde (GA) | 2.8 | < | 9 | 2.6 | < |
| Dihydroxyacetone (DHA) | 2.5 | < | 6 | 2.2 | < |
| Hydroxyacetone (HA) | 2.3 | 3.1 | < | < | < |
| Malondialdehyde | < | < | < | 15 | |
| Glyoxal/benzaldehyde | < | < | < | < | |
| Hexanal | < | < | < | < | |
| Octanal | < | < | < | < | |
| Decanal | < | < | < | < | |
| Dodecanal | < | < | < | < | |
| Tetradecanal | < | < | < | < | |
| Hexadecanal | < | < | < | < | |
| Total aldehydes/ketones | 13.6 | 3.1 | 24.6 | 8.6 | 0 |
| Total GA, DHA, HA | 7.6 | 3.1 | 15 | 4.8 | 0 |

| | F | G | H | I |
|---|---|---|---|---|
| Substance | ppm | ppm | ppm | ppm |
| Formaldehyde | < | < | < | < |
| Acetaldehyde | < | < | < | < |
| Acrolein | < | < | < | < |
| Acetone | < | < | < | < |
| Propanal | < | < | < | < |
| Butanal | < | < | < | < |
| Glyceraldehyde (GA) | < | < | 2.5 | < |
| Dihydroxyacetone (DHA) | < | < | < | < |
| Hydroxyacetone (HA) | < | < | < | < |
| Malondialdehyde | < | < | < | < |
| Glyoxal/benzaldehyde | < | < | < | < |
| Hexanal | < | < | < | < |
| Octanal | < | < | < | < |
| Decanal | < | < | < | < |
| Dodecanal | < | < | < | < |
| Tetradecanal | < | < | < | < |
| Hexadecanal | < | < | < | < |
| Total aldehydes/ketones | 0 | 0 | 2.5 | 0 |
| Total GA, DHA, HA | 0 | 0 | 2.5 | 0 |

Result:

The hydrogenation clearly reduces the aldehydes and ketones present. No new oxidized secondary components were formed in the subsequent distillation step.

### Example 9

### Dependence on Concentration of Reduction with Borohydride in Glycerol

10% water and equal parts of formaldehyde, propionaldehyde, glyceraldehyde, dihydroxyacetone and hydroxyacetone were added to pure glycerol. The sum of detectable aldehydes amounted to 152 ppm. Borohydride solution was added in concentrations of 500 ppm, 1500 ppm and 5,000 ppm, followed by incubation for 2 h at 180° C. The mixtures were then adjusted to pH 5 with citric acid and analyzed.

Result:

Even at a concentration of 500 ppm, the aldehydes and ketones were quantitatively reduced and were no longer detectable.

### Example 10

### Dependence on Concentration of Reduction with Borohydride in Glycerol

10% water and equal parts of formaldehyde, propionaldehyde, glyceraldehyde, dihydroxyacetone and hydroxyacetone were added to a pure glycerol of relatively poor quality. The sum of detectable aldehydes amounted to 44 ppm. Borohydride solution was added in concentrations of 400 ppm, 1,000 ppm, 2,000 ppm and 5,000 ppm, followed by incubation for 2 h at 180° C. The mixtures were then adjusted to pH 5 with hydrochloric acid and analyzed.

Result:

Aldehydes and ketones were detectable up to a concentration of 1,000 ppm; from 2,000 ppm borohydride solution, the aldehydes and ketones were quantitatively reduced and were no longer detectable.

### Example 11

### Dependence on Concentration of Reduction with Borohydride in Glycerol

500 ppm and 2,000 ppm borohydride solution were added to methanol-containing crude glycerol from an alkaline low-pressure transesterification with a total content of detectable aldehydes and ketones of 188 ppm, followed by incubation for 2 h at 80° C. The methanol was then distilled off and the crude glycerol was analyzed.

Result:

Even with 2,000 ppm borohydride solution, no significant reduction in the aldehyde and ketone concentration could be detected by comparison with the untreated sample.

### Example 12

### Influence of Water Content on Reduction with Borohydride in Glycerol

The borohydride treatment of pure glycerol was carried out in the same way as in Example 9, except that no water was added to the glycerol and the aldehydes and ketones were directly dissolved in the glycerol. A total concentration of 114 ppm aldehydes and ketones was detected in the pure glycerol.

Result:

Even in water-free glycerol, all aldehyde and ketones were quantitatively reduced from a concentration of 500 ppm borohydride solution.

### Example 13

### Comparative Analysis of Examples 9 to 12

The purity of the glycerol has a strong influence on the effect of the borohydride. The more impurities present in the glycerol, the poorer the effect of the borohydride which is attributable to chemical compounds that consumed the borohydride.

Accordingly, high-quality starting glycerols are the most suitable for the following reductive purification.

The water content of the glycerol does not have a significant effect on the boron-hydride-catalyzed reduction.

### Example 14

### Formation of Aldehydes and Ketones in the Treatment of Glycerol with Silicates

0.1% silicate (Trisyl™) was added to a pure glycerol, followed by incubation for 1 hour at 60° C. 0.1% of another silicate (Magnesol™) was added to the same pure glycerol, followed by incubation for 1 hour at 140° C. After the silicate treatment, the silicates were removed from the glycerol by filtration and the glycerols were compared with the starting substance as reference for their aldehyde and ketone contents.

| | A | B | C |
|---|---|---|---|
| Substance | ppm | ppm | ppm |
| Formaldehyde | < | 0.5 | 6.6 |
| Acetaldehyde | < | < | 0.5 |
| Acrolein | < | < | 4.9 |
| Acetone | < | < | 4.3 |
| Propanal | < | < | 2.7 |
| Butanal | < | < | 0.5 |
| Glyceraldehyde (GA) | 4.4 | 5.1 | 6.2 |
| Dihydroxyacetone (DHA) | 3.6 | 3.4 | 9.2 |
| Hydroxyacetone (HA) | < | < | 61 |
| Malondialdehyde | < | < | < |
| Glyoxal/benzaldehyde | < | < | 10 |
| Hexanal | < | < | < |
| Octanal | < | < | < |
| Decanal | < | < | < |
| Dodecanal | < | < | < |
| Tetradecanal | < | < | < |
| Hexadecanal | < | < | < |
| Total aldehydes/ketones | 8 | 9 | 105.9 |
| Total GA, DMA, HA | 8 | 8.5 | 76.4 |

Result:

The treatment with activated silicates at low temperature is suitable for the removal of metal traces. By contrast, a silicate treatment at high temperatures has a very adverse effect on the concentration of aldehydes and ketones.

### Example 15

### Oxidation Stability of Various Crude Glycerols During Storage by Comparison With Glycerol Produced from Propylene

Crude glycerols from Example 2, samples A and C, were subjected to a storage test. For this test, the samples were bottled in glass bottles and plastic bottles (the glass bottles were the standard transparent glass bottles used for storing chemicals in the laboratory) and stored at a constant temperature of 40° C. (in darkness in a heating cabinet). Every four weeks, a sample was taken and analyzed. The following Table shows the sum of glyceraldehyde and dihydroxyacetone which are the direct oxidation products starting from glycerol:

A: pure glycerol from chemical production (from Example 4, sample A);

B: pure glycerol from enzymatic splitting (from Example 2, sample A);

C: pure glycerol from alkaline trasesterification (Example 2, sample C).

| | Start | 4 Weeks | 4 Weeks | 8 Weeks | 8 Weeks |
|---|---|---|---|---|---|
| | Storage | Plastic | Glass | Plastic | Glass |
| Sample | ppm GA + DHA | ppm GA + DHA | ppm GA + DHA | ppm GA + DHA | ppm GA + DHA |
| A | 0 | 2.6 | 2.6 | 9.6 | 9.2 |
| B | 9.1 | 34 | 23 | 54 | 43 |
| C | 27 | 34 | 76 | 51 | 26 |

Result:

The crude glycerols show greater aldehyde and ketone formation than the pure glycerol.

### Example 16

### Oxidation Stability of Pure Glycerols During Storage by Comparison with Glycerol Produced from Propylene

Pure glycerols from Example 4, samples A to C, were subjected to a storage test. For this test, the samples were bottled in glass bottles and plastic bottles and stored at a constant temperature of 40° C. Every four weeks, a sample was taken and analyzed. The following Table shows the sum of glyceraldehyde and dihydroxyacetone which are the direct oxidation products starting from glycerol:

| | Start | 4 Weeks | 4 Weeks | 8 Weeks | 8 Weeks |
|---|---|---|---|---|---|
| | Storage | Plastic | Glass | Plastic | Glass |
| Sample | ppm GA + DHA | ppm GA + DHA | ppm GA + DHA | ppm GA + DHA | ppm GA + DHA |
| A | 0 | 2.6 | 2.6 | 9.6 | 9.2 |
| B | 0 | 6.3 | 6.1 | 10.3 | 9.9 |
| C | 2.5 | 7.7 | 8.3 | 12.8 | 11.9 |

Result:

Even the pure glycerols were not stable in storage. A clear increase in the aldehydes and ketones was observed over 8 weeks' storage.

### Example 17

### Oxidation Stability of Hydrogenated Crude Glycerol During Storage by Comparison with Untreated Crude Glycerol

The hydrogenated crude glycerols from Example 5 were compared with the corresponding starting glycerols in the storage test:

A: crude glycerol from enzymatic hydrolysis;

B: sample A hydrogenated and distilled;

C: crude glycerol from alkaline low-pressure transesterifaction;

D: sample C hydrogenated and distilled.

| | Start | 4 Weeks | 4 Weeks | 8 Weeks | 8 Weeks |
|---|---|---|---|---|---|
| | Storage | Plastic | Glass | Plastic | Glass |
| Sample | ppm GA + DHA | ppmGA + DHA | ppm GA + DHA | ppm GA + DHA | ppm GA + DHA |
| A | 27 | 34 | 76 | 51 | 26 |
| B | 4.8 | 7.2 | 7.8 | 9.2 | 9.4 |
| C | 9.1 | 34 | 23 | 54 | 43 |
| D | 0 | 0 | 0 | 7.8 | 2.2 |

Result:

The storage stability of the hydrogenated crude glycerols is clearly improved.

### Example 18

### Oxidation Stability of Hydrogenated Pure Glycerol During Storage by Comparison with Untreated Pure Glycerol

The hydrogenated pure glycerols from Example 5 were compared with the corresponding starting glycerols in the storage test:

A: pure glycerol from chemical production (Example 4, sample A);

B: vegetable-based pure glycerol (Example 4, sample B);

C: sample B hydrogenated and distilled;

D: sample B hydrogenated, acidified and distilled;

E: vegetable-based pure glycerol (Example 4, sample C);

F: sample E hydrogenated and distilled.

Result:

The storage stability of the hydrogenated pure glycerols was clearly improved. After 8 weeks, little, if any, formation of aldehydes and ketones was observed. The hydrogenated vegetable glycerols were of better quality than pure glycerol produced from propylene.

| | Start | 4 Weeks | 4 Weeks | 8 Weeks | 8 Weeks |
|---|---|---|---|---|---|
| | Storage | Plastic | Glass | Plastic | Glass |
| Sample | ppm GA + DHA | ppm GA + DHA | ppm GA + DHA | ppm GA + DHA | ppm GA + DHA |
| A | 0 | 2.6 | 2.6 | 9.6 | 9.2 |
| B | 0 | 6.3 | 5.1 | 10.3 | 9.9 |
| C | 0 | 0 | 0 | 0 | 0 |
| D | 0 | 0 | 0 | 0 | 0 |
| E | 2.5 | 7.7 | 8.3 | 12.8 | 11.9 |
| F | 0 | 0 | 0 | .0 | 2.6 |

### Example 19

### Oxidation Stability of Silicate-Treated Pure Glycerol During Storage by Comparison with Untreated Pure Glycerol

The samples from Example 14 were subjected to a storage test.

| | Start | 4 Weeks | 4 Weeks | 8 Weeks | 8 Weeks |
|---|---|---|---|---|---|
| | Storage | Plastic | Glass | Plastic | Glass |
| Sample | ppm GA + DHA | ppm GA + DHA | ppm GA + DHA | ppm GA + DHA | ppm GA + DHA |
| A | 7.7 | 12 | 13.3 | 18.7 | 18.7 |
| B | 8.5 | 13.7 | 13.7 | 23 | 23 |
| C | 15.4 | 77 | 56 | 115 | 118 |

Result:

The treatment with silicates at low temperature had no significant negative effect on the re-formation of aldehydes and ketones. To this extent, the treatment appears to be suitable for removing metal traces in conjunction with a borohydride reduction.

### Example 20

### Oxidation Stability of Soap-Spiked Pure Glycerol During Storage by Comparison with Untreated Pure Glycerol

200 ppm potassium linolenate were mixed with pure glycerol from Example 4, sample B. The glycerol spiked with polyunsaturated soap (sample B) was compared with the starting substance (sample A) in the storage test.

| | Start | 4 Weeks | 4 Weeks | 8 Weeks | 8 Weeks |
|---|---|---|---|---|---|
| | Storage | Plastic | Glass | Plastic | Glass |
| Sample | ppm GA + DHA | ppm GA + DHA | ppm GA + DHA | ppm GA + DHA | ppm GA + DHA |
| A | 0 | 6.3 | 6.1 | 10.3 | 9.9 |
| B | 2.1 | 7.7 | 9.3 | 16.7 | 19.8 |

Result:

Polyunsaturated soap catalyzed an increased re-formation of aldehydes and ketones.

1) A process for the production of glycerol comprising the steps of:
(a) providing a vegetable oil or fat as starting material;
(b) obtaining crude glycerol from the vegetable oil or fat; and
(c) treating the crude glycerol with a reducing agent.

2) (canceled)

3) The process of claim 1 wherein the treated glycerol of (c) has an aldehyde and ketone content of less than 9 ppm.

4) The process of claim 3 wherein the aldehyde is glyceraldehyde and the ketone is dihydroxyacetone.

5) The process of claim 1 wherein the reducing agent is a hydrogenating agent.

6) The process of claim 5 wherein the hydrogenating agent is a borohydride.

7) The process of claim 6 wherein the borohydride is sodium borohydride.

8) The process of claim 7 wherein sodium borohydride is used in an amount of from 0.005 to 0.05% by weight, based on the crude glycerol to be treated.

9) The process of claim 1 which further comprises distilling the crude glycerol prior to treating the crude glycerol with the reducing agent.

10) The process of claim 6 which further comprises distilling the treated glycerol to remove boron.

11) The process of claim 1 wherein the crude glycerol to be treated is obtained from the vegetable oil or fat in a process carried out at a temperature below 100° C. and at a pressure below 2 bar.

12) The process of claim 1 which further comprises treating the glycerol with a silicate either before or after treating the glycerol with the reducing agent.

13) The glycerol produced by the process of claim 1.

14) The glycerol of claim 13 which further comprises a pharmaceutically active ingredient.

15) The glycerol produced by the process of claim 1 in which the glycerol, after storage in the dark at 40° C. for 8 weeks, has a total content of glyceraldehyde and dihydroxyacetone of less than 9 ppm.
